(19) United States
(12) Patent Application Publication
(10) Pub. No.: US 2018/0226156 A1
(43) Pub. Date: Aug. 9, 2018

(54) METHODS FOR ASSESSING PHARMACEUTICAL PERFORMANCE ACROSS THERAPEUTIC AREAS AND DEVICES THEREOF

(71) Applicant: BioXcel Corporation, Branford, CT (US)

(72) Inventors: Krishnan NANDABALAN, Guilford, CT (US); Vimal MEHTA, Guilford, CT (US)

(21) Appl. No.: 15/746,662

(22) PCT Filed: Jul. 22, 2015
(86) PCT No.: PCT/US2015/041460

§ 371 Date: Jan. 22, 2018

(51) Int. Cl. G16H 70/40

(52) U.S. Cl. CPC G16H 70/40

(57) ABSTRACT

A method, non-transitory computer readable medium, and pharmaceutical assessment computing device that effectively assesses pharmaceutical data. With this technology pharmaceutical data for a candidate drug is obtained. The pharmaceutical data including a drug indication and a pharmaceutical value for a plurality of pharmaceutical parameters. Next, standard of care data including standard of care values corresponding to the obtained drug indication for the candidate drug is obtained and deviations between the pharmaceutical parameter values and the standard of care values is determined. A plurality of pharmaceutical assessment scores is assigned to each one of a plurality of pharmaceutical parameters based on the determined deviations. A target product profile for the candidate drag is provided based on the assigned plurality of pharmaceutical assessment scores.

### FIELD

This technology relates to methods, computing devices, and non-transitory computer readable media that assess clinical, preclinical, and market performance of pharmaceuticals across multiple therapeutic areas.

### BACKGROUND

The drug development process is fraught with impediments, ranging from the high costs associated with research and development to various regulatory hurdles that must be overcome in order to receive government approval to conduct clinical trials. Though clinical trials often last years, conducting trials does not guarantee a clinically successful drug, let alone a drug with the potential to be profitable. Further, the costs associated with making a drug commercially viable do not end with the development and testing of the drug, but also include subsequent expenses relating to the production and marketing of the finished drug product. As a result, a need exists in the pharmaceutical industry for tools to aid in the assessment of pharmaceutical data and to mitigate the risks, both medical and commercial, that are associated with the development of new drugs.

Current software tools for the assessment of pharmaceuticals include a variety of ways to access pharmaceutical information sources such as websites and health related databases, as well as more traditional sources of information like journals. Additionally, these software tools facilitate access to both general information as well as specialized information sources directed towards medical professionals. However, this sea of pharmaceutical information is largely disorganized or organized along the broadest lines, offering no specific way to compare a candidate drug in development to the disparate pharmaceuticals that are currently undergoing clinical trials or are already available on the market. As a result, a great deal of time and substantial resources are used to assess the potential impact of a candidate drug, both in terms of its pharmaceutical performance and its potential as a commercial product.

Accordingly, there is a need for improved software technology that is able to gather and organize pharmaceutical information in a manner that provides more effective assessment of pharmaceutical data. Further, there is a need for improved software technology that is able to provide organized pharmaceutical information in an accessible format that may be used to more accurately assess the potential viability of a candidate drug.

### SUMMARY

A method for assessing pharmaceutical performance comprising, obtaining, by a pharmaceutical assessment computing device, pharmaceutical data for a candidate drug, the pharmaceutical data comprising a drug indication and a pharmaceutical parameter value for each of a plurality of pharmaceutical parameters. The pharmaceutical assessment computing device, obtains, for the drug indication, standard of care data comprising a standard of care value for each of the plurality of pharmaceutical parameters. The pharmaceutical assessment computing device determines a deviation of each of the pharmaceutical parameter values from the corresponding one of the standard of care values. Next, the pharmaceutical assessment computing device assigns one of a plurality of pharmaceutical assessment scores to each of the plurality of pharmaceutical parameters based on each of the determined deviations. The pharmaceutical assessment computing device provides target product profile data for the candidate drug based on the assigned plurality of pharmaceutical assessment scores.

A pharmaceutical assessment computing device comprising at least one processor and a memory, wherein the memory is coupled to the processor which is configured to be capable of executing programmed instructions, which comprise the programmed instructions stored in the memory to: obtain pharmaceutical data for a candidate drug, the pharmaceutical data comprising a drug indication and a pharmaceutical parameter value for each of a plurality of pharmaceutical parameters. The pharmaceutical assessment computing device, obtains, for the drug indication, standard of care data comprising a standard of care value for each of the plurality of pharmaceutical parameters. The pharmaceutical assessment computing device determines a deviation of each of the pharmaceutical parameter values from the corresponding one of the standard of care values. Next, the pharmaceutical assessment computing device assigns one of a plurality of pharmaceutical assessment scores to each of the plurality of pharmaceutical parameters based on each of the determined deviations. The pharmaceutical assessment computing device provides target product profile data for the candidate drug based on the assigned plurality of pharmaceutical assessment scores.

A non-transitory computer-readable medium having stored thereon instructions for assessing pharmaceutical data, comprising machine executable code which when executed by at least one processor, causes the processor to perform steps comprising obtaining pharmaceutical data for a candidate drug, the pharmaceutical data comprising a drug indication and a pharmaceutical parameter value for each of a plurality of pharmaceutical parameters. Standard of care data is obtained for the drug indication, the standard of care data comprising a standard of care value for each of the plurality of pharmaceutical parameters. A deviation of each of the pharmaceutical parameter values from the corresponding one of the standard of care values is determined. One of a plurality of pharmaceutical assessment scores is assigned to each of the plurality of pharmaceutical parameters based on each of the determined deviations. Target product profile data is provided for the candidate drug based on the assigned plurality of pharmaceutical assessment scores.

This technology offers a number of advantages including providing more effective methods, devices, and non-transitory computer readable media for assessing pharmaceutical data. By way of example only, with this technology, pharmaceutical data for a candidate drug may be compared against standard of care data in order to provide immediate feedback on whether the candidate drug meets safety standards such as those mandated by government regulatory agencies. Further, this technology provides a streamlined way to produce a target product profile, thereby facilitating drug development.

### BRIEF DESCRIPTION OF THE DRAWINGS

FIG. 1 is a block diagram of a network environment with an exemplary pharmaceutical assessment computing device;

FIG. 2 is a block diagram of the exemplary pharmaceutical assessment computing device;

FIG. 3 is a flow chart of an example of a method for assessing pharmaceutical data with the exemplary pharmaceutical assessment computing device;

FIG. 4 is a table of biomarker categories generated by the pharmaceutical assessment computing device;

FIG. 5 is a graph of a comparison between a candidate drug and standard of care drugs generated by the exemplary pharmaceutical assessment computing device;

FIG. 6 is a map of a disease process generated by the exemplary pharmaceutical assessment computing device;

FIG. 7 is a table illustrating an exemplary target product profile generated by the exemplary pharmaceutical assessment computing device; and

FIG. 8 is a timing diagram of an example of a method for assessing pharmaceutical data between a pharmaceutical assessment computing device, a server computing device, and a client computing device.

### DETAILED DESCRIPTION

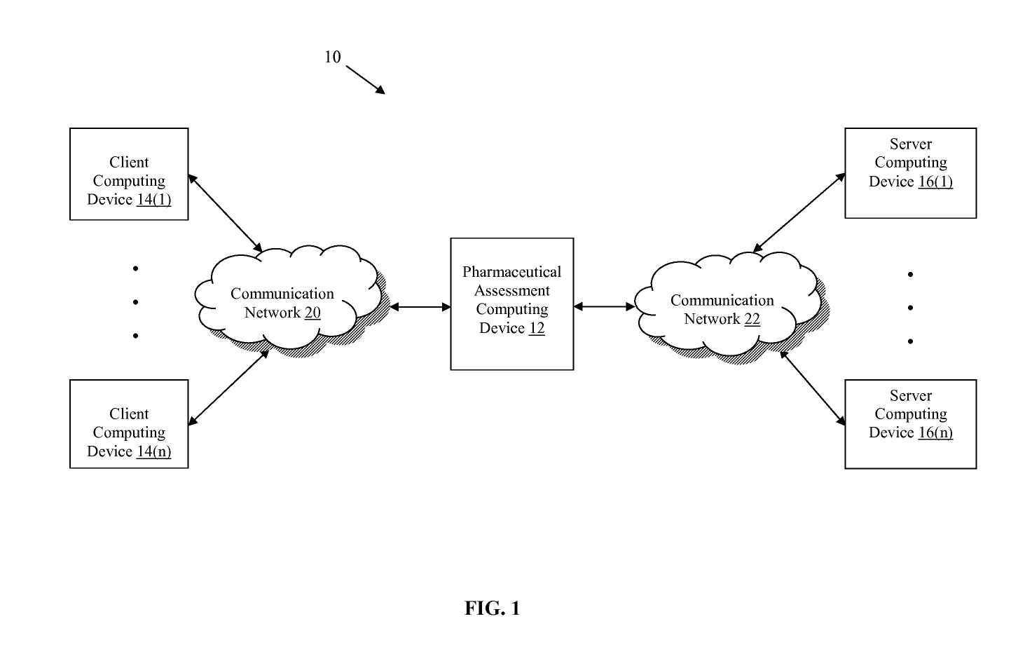

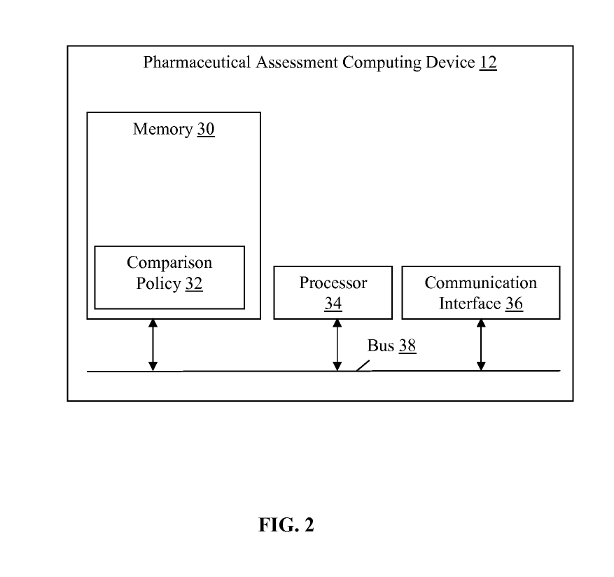

An example of a network environment 10 with an example of a pharmaceutical assessment computing device 12 that assesses pharmaceutical data is illustrated in FIGS. 1-2. In this particular example, the network environment 10 includes the pharmaceutical assessment computing device 12, a plurality of client computing devices 14(1)-14(n), and a plurality of server computing devices 16(1)-16(n), which are coupled together by communication network 20 and communication network 22, although the network environment 10 can include other types and/or numbers of other systems, devices, components, and/or other elements in other configurations. Further, the network environment 10 may include other network devices such as one or more routers or switches, for example, which are known to those skilled in the art and will not therefore be described here. This technology provides a number of advantages including methods, non-transitory computer readable media, and computing devices that perform assessment of pharmaceutical data.

Referring to FIGS. 1-2, the pharmaceutical assessment computing device 12 is illustrated as coupled to communication network 20, though pharmaceutical assessment computing device 12 may be coupled to other types or numbers of communication networks. The pharmaceutical assessment computing device 12 may perform any number of functions including determining a mechanism of action, estimating clinical performance, and providing a target product profile data based on candidate drug data. The pharmaceutical assessment computing device 12 in this example includes a processor 34, a memory 30, and a communication interface 36 which are coupled together by one or more bus 38 or other links, although the pharmaceutical assessment computing device 12 may include other types or numbers of elements in other configurations. In this example, the bus 38 is a hyper-transport bus, although other bus types and communication links may be used, such as Peripheral Component Interconnect (PCI).

The processor 34 of the pharmaceutical assessment computing device 12 may execute one or more programmed instructions stored in the memory 30 as illustrated and described in the examples herein, although other types and numbers of functions or other operation can be performed. The processor of the pharmaceutical assessment computing device 12 may include one or more central processing units (CPUs) or general purpose processors with one or more processing cores.

The memory 30 of pharmaceutical assessment computing device 12 stores the programmed instructions and other data for one or more aspects of the present technology as described and illustrated herein, although some or all of the programmed instructions could be stored and executed elsewhere. A variety of different types of memory storage devices, such as a random access memory (RAM) or a read only memory (ROM) in the system or a floppy disk, hard disk, CD ROM, DVD ROM, or other computer readable medium which is read from and written to by a magnetic, optical, or other reading and writing system that is coupled to the processor, can be used for the memory.

In this example, the memory 30 of the pharmaceutical assessment computing device 12 includes a comparison policy 32 that may be used to compare various data such as pharmaceutical data or standard of care data. The comparison policy 32 may be used to evaluate pharmaceutical data comprising characteristics of a candidate drug against one or more standard of care drugs and further to apply a weighting factor to each one of a plurality of pharmaceutical values in a plurality of pharmaceutical parameters included in the pharmaceutical data.

The communication interface 36 of the pharmaceutical assessment computing device 12 operatively couples and communicates between the pharmaceutical assessment computing device 12, client computing devices 14(1)-14(n), and server computing devices 16(1)-16(n), which are all coupled together by the communication network 20 or communication network 22, although other types and numbers of communication interfaces and connections and configurations to other equipment, systems or devices may be used.

By way of example only, the communication network 20 or communication network 22 can use Transmission Control Protocol/Internet Protocol (TCP/IP) over Ethernet and industry-standard protocols, although other types and numbers of communication networks can be used. Communication network 20 or communication network 22 in this example may employ any suitable interface mechanisms and network communication technologies including, for example, teletraffic in any suitable form (e.g., voice, modem, and the like), Public Switched Telephone Network (PSTNs), Ethernet-based Packet Data Networks (PDNs), combinations thereof, and the like.

Each of the client computing devices 14(1)-14(n) includes a processor, a memory, and a communication interface, which are coupled together by a bus or other link, although other numbers and types of network devices could be used. The client computing devices 14(1)-14(n) may run interface applications that may provide an interface to make requests for authentication, authorization, and accounting services for users, for example, via the communication network 20 and the pharmaceutical assessment computing device 12.

Each of the server computing devices 16(1)-16(n) includes a processor, a memory, and a communication interface, which are coupled together by a bus or other link, although other numbers and types of network devices could be used. The server computing devices 16(1)-16(n) may be hardware or software or may represent a system with multiple servers in a server computing device pool, which may include internal or external networks.

Although the exemplary network environment 10 with the pharmaceutical assessment computing device 12, client computing devices 14(1)-14(n), and server computing devices 16(1)-16(n), communication network 20, and communication network 22 are described and illustrated herein, other types and numbers of systems, devices, components, and elements in other topologies can be used. The systems of the examples described herein are for exemplary purposes, as many variations of the specific hardware and software used to implement the examples are possible, as will be appreciated by those skilled in the relevant art(s).

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only teletraffic in any suitable form (e.g., voice and modem), wireless traffic media, wireless traffic networks, cellular traffic networks, G3 traffic networks, Public Switched Telephone Network (PSTNs), Packet Data Networks (PDNs), the Internet, intranets, and combinations thereof.

The examples may also be embodied as a non-transitory computer readable medium having instructions stored thereon for one or more aspects of the present technology as described and illustrated by way of the examples herein, which when executed by the processor, cause the processor to carry out the steps necessary to implement the methods of this technology as described and illustrated with the examples herein.

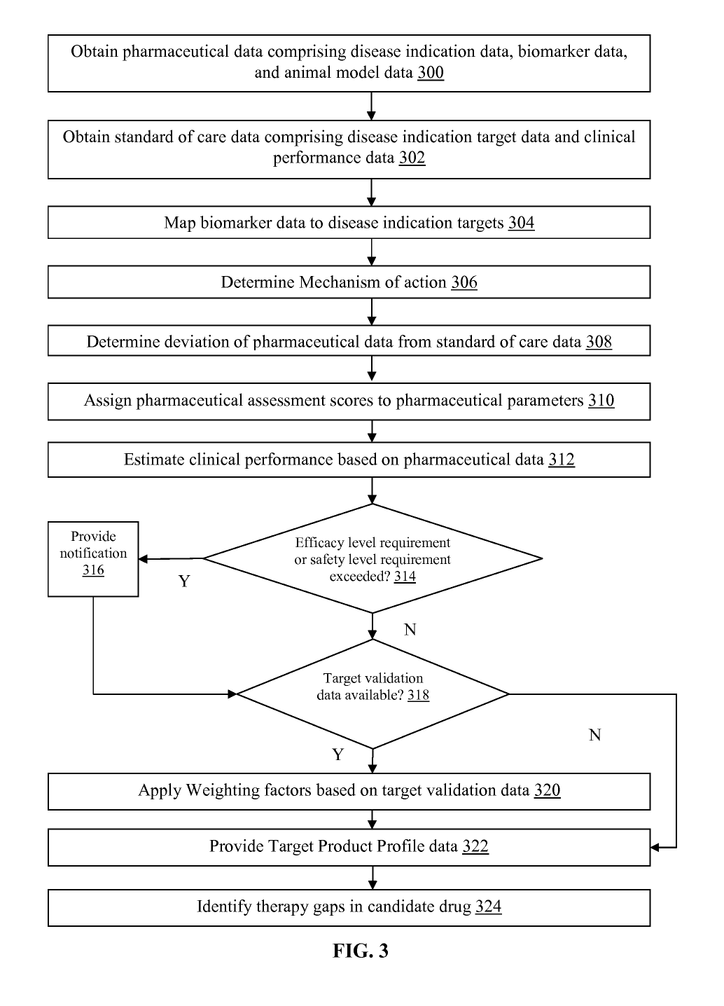

An example of a method for assessing pharmaceutical data will now be described with reference to FIGS. 1-8. Referring more specifically to FIG. 3, an exemplary method for assessing pharmaceutical data will now be described. In step 300 in this example, a pharmaceutical assessment computing device 12 obtains pharmaceutical data for a candidate drug from server computing devices 16(1)-16(n). Alternatively, pharmaceutical assessment computing device 12 may communicate through communication network 22 to obtain pharmaceutical data from a number of data sources such as PubMed, the National Center for Biotechnology information (NCBI), the National Institutes for Health (NIH), Medline, Scirus, and other repositories of medical or pharmaceutical data.

The pharmaceutical data in this example includes drug indication data for a levodopa derivative candidate drug, intended for use in the treatment of Parkinson's disease. Further, the pharmaceutical data also includes a plurality of pharmaceutical parameters relating to the Parkinson's disease candidate drug, in which each one of the plurality of pharmaceutical parameters has a corresponding pharmaceutical value.

In this example, the pharmaceutical parameters and corresponding pharmaceutical values may include epidemiological data, such as the gender distribution, average age of onset, prevalence, incidence, and 5-year mortality of patients diagnosed with Parkinson's disease. In an alternative example, the drug indication data may comprise data related to disease treatment, pain management, or other therapies associated with medical treatment and the plurality of pharmaceutical parameters and corresponding pharmaceutical values may include data relating to the chemical or biological characteristics of the candidate drug and its testing and use in a medical treatment including discovery-stage data, preclinical data, Phase I clinical trials data, Phase II clinical trials data, Phase III clinical trials data, formulation data, formulation manufacturing data, or pharmacovigilance data relating to the detection, assessment, and prevention of adverse effects associated with a drug.

In step 302, the pharmaceutical assessment computing device 12 obtains standard of care data for the Parkinson's disease drug indication, from server computing devices 16(1)-16(n). In this example, the standard of care data includes data for drugs that are approved for treatment of Parkinson's disease by a governmental drug regulatory agency such as the United States Food and Drug Administration (FDA), European Medicines Agency (EMA), or the Japanese Ministry of Health and Welfare, though alternative examples include standard of care data that is not limited to drugs that are approved by a governmental regulatory agency.

In this example, the standard of care data also includes a set of standard of care parameters and respective standard of care pharmaceutical values that correspond to the pharmaceutical parameters and pharmaceutical values obtained in step 302, including epidemiological data such as the gender distribution, average age of onset, prevalence, incidence, and 5-year mortality of Parkinson's disease as well as the pharmaceutical data relating to the candidate drug, chemical or biological characteristics of the candidate drug, and its testing and use in a medical treatment including discovery-stage data, preclinical data, Phase I clinical trials data, Phase II clinical trials data, Phase III clinical trials data, formulation data, formulation manufacturing data, or pharmacovigilance data relating to the detection, assessment, and prevention of adverse effects associated with a drug.

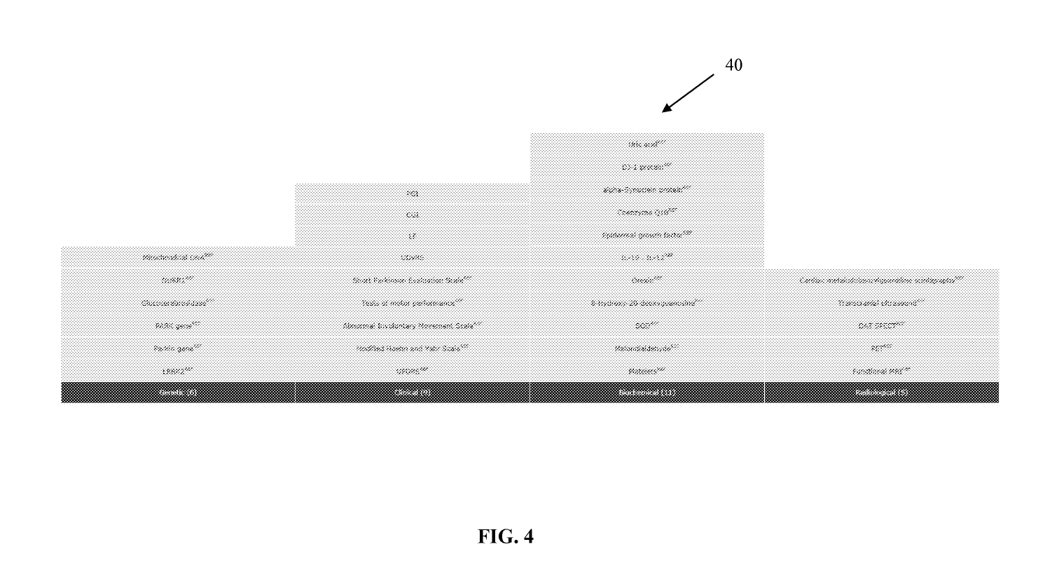

In step 304, the pharmaceutical assessment computing device 12 maps biomarker data for the candidate drug to one or more drug indication target parameters. In this example, the biomarker data is mapped to drug indication target parameters including genetic data parameters, clinical data parameters, biochemical data parameters, and radiological data parameters for the candidate drug as illustrated in biomarker table 40 in FIG. 4. Each one of the drug indication target parameters includes further data on the specific biomarker that was mapped.

For example the gene LRRK2 (Leucine-Rich Repeat Kinase 2), mapped to the genetic data drug indication target parameter, is a biomarker for mutations in LRRK2 that are associated with Parkinson's disease. The clinical data drug indication target parameter is mapped to clinical biomarker data including data relating to the UPDRS (Unified Parkinson's Disease Rating Scale) which is used to assess data on the longitudinal course of Parkinson's disease based on clinical observations and data collected from patient interviews. The biochemical data drug indication target parameter is mapped to biochemical biomarker data including the alpha-synuclein protein data and includes pharmacological data indicating how the candidate drug helps synaptic vesicle transport, a correlation of the significance of the alpha-synuclein protein to the Parkinson's disease. The radiological drug indication target parameter is mapped to radiological biomarker data including a DAT SPECT (Dopamine Transport Single Photon Emission Computed Tomography) biomarker that provides data that can aid in confirming a diagnosis of Parkinson's disease.

In an alternative example, the pharmaceutical assessment computing device 12 is able to filter biomarkers based on whether the biomarker is approved by a drug regulatory agency such as the Food and Drug Administration (FDA). For example, the DAT SPECT radiological biomarker to measure neuronal loss is approved by the FDA, whereas the transcranial ultrasound biomarker is not FDA approved. In this way, the pharmaceutical assessment computing device 12 is able to more effectively determine a target product profile by ensuring that only FDA approved biomarkers are included in the assessment of whether a candidate drug meets a target product profile.

In step 306, the pharmaceutical assessment computing device 12 determines at least one mechanism of action for the candidate drug, based on the mapped biomarker data. In this example, the pharmaceutical assessment computing device 12 determines that a mechanism of action for the candidate drug is a DR (Dopamine Receptor) agonist, based on the candidate drug biomarker information which includes data indicating that the candidate drug is a levodopa derivative for which a DR agonist is a corresponding mechanism of action. In an alternative example, the candidate drug may be associated with more than one mechanism of action and the mechanism of action may be used as a basis for comparison against other drugs. The comparison against other drugs may further include a comparison of portfolios associated with the makers of competing drugs or a strategic intent map of associated drug indications across a therapeutic area for a particular disease.

In step 308, the pharmaceutical assessment computing device 12 determines a deviation, for the drug indication, of the pharmaceutical data from the standard of care data. In this example each one of the pharmaceutical parameter values is compared to a corresponding standard of care parameter value. For example, a pharmaceutical parameter for the levodopa level in a group being treated with the candidate drug has a pharmaceutical parameter value for levodopa level of 800 ng/ml. This pharmaceutical parameter value for levodopa level is compared to a corresponding standard of care parameter value for levodopa level in a group being treated with a placebo with a corresponding levodopa level of 160 ng/ml, yielding a deviation of 640 ng/ml.

In the preceding example, a significant (640 ng/ml) deviation in levodopa level between the pharmaceutical data value and the standard of care data value may be an important indicator of the efficacy of the candidate drug. The pharmaceutical assessment computing device 12 will than iterate through the remaining pharmaceutical parameters and compare the pharmaceutical parameter values to corresponding standard of care parameter values in order to determine a deviation value for each one of the remaining pharmaceutical parameters.

In an alternative example, the deviation value for each one of the pharmaceutical parameter values may be a standard deviation of the pharmaceutical parameter value from the standard of care value, a proportional value such as a percentile value, or a relative ranking. Further, the pharmaceutical data may be compared to multiple standard of care drugs included in the standard of care data and deviation values may be determined for each standard of care drug individually. Additionally, the comparison between the pharmaceutical data and the standard of care data may include an indication of the trial confidence for any one of the drugs included in the pharmaceutical data or standard of care data.

In step 310, the pharmaceutical assessment computing device 12 assigns a plurality of pharmaceutical assessment scores to each one of the plurality of pharmaceutical parameter values based on the determined deviation values. In this example, the pharmaceutical score for each one of the plurality of pharmaceutical parameters is represented by a value on a numerical scale ranging from zero to five. Using the determined deviation from the levodopa level pharmaceutical parameter value, a pharmaceutical score of five is assigned to the respective pharmaceutical parameter to reflect the magnitude of the deviation between the pharmaceutical parameter value and the standard of care parameter value.

In the example of the levodopa level pharmaceutical parameter, the assigned pharmaceutical assessment score corresponds to the absolute difference (five-fold) in magnitude between the pharmaceutical parameter value and the standard of care value. In an alternative example the relationship between the assigned pharmaceutical assessment score and the determined deviation may be based on different relationships including exponential relationships, or logarithmic relationships.

In step 312, the pharmaceutical assessment computing device 12 estimates clinical performance of the candidate drug based on the pharmaceutical data. In this example, the estimate of clinical performance includes two scores: a potential to address ideal target product profile score which aggregates the pharmaceutical assessment scores for the candidate drug and measures the aggregated score against an ideal target product profile score based on the standard of care data; and a potential to achieve regulatory success score also based on the aggregated standard of care data.

In an alternative example, the estimate of clinical performance may form the basis for estimating additional performance indicators for the candidate drug, such as an expected market entry scenario, expected patient share forecast, or potential commercial viability.

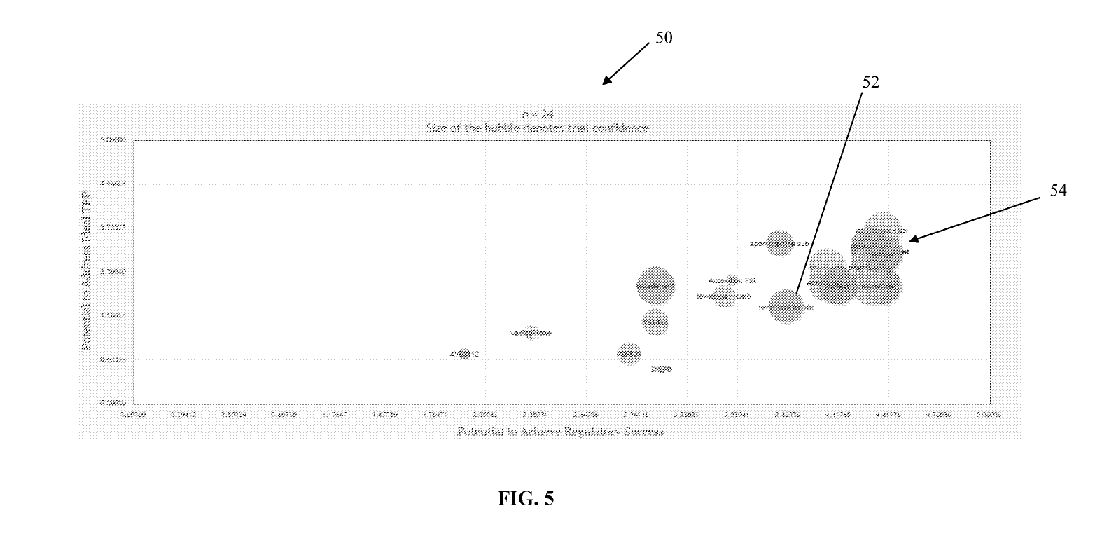

FIG. 5 illustrates a comparison graph 50 showing a comparison between levodopa inhale 52 and a plurality of Parkinson's disease drugs 54. In comparison graph 50, the pharmaceutical assessment scores have been aggregated into two different scores represented by a vertical axis indicating the potential to address ideal target product profile score and a horizontal axis indicating the potential to achieve regulatory success axis, illustrating an estimate based on two separate sets of accumulated pharmaceutical assessment scores. As illustrated in FIG. 5, the size of the bubble corresponding to each of the plurality of Parkinson's disease drugs 54 denotes the trial confidence for the respective drug. In an alternative example, the pharmaceutical assessment scores may be based on different estimates of clinical performance such as the potential to achieve regulatory success within a predetermined time period or the potential to achieve an ideal target product profile score exceeding a predetermined threshold level.

In step 314, the pharmaceutical assessment computing device 12 determines whether an efficacy requirement or a safety level requirement is satisfied. In this example a Unified Parkinson's Disease Rating Scale (UPDRS) Part III parameter value is used to determine whether the efficacy requirement is satisfied, though in alternative examples, a different combination of the one or more pharmaceutical parameter values may be used. The UPDRS Part III pharmaceutical parameters comprise clinician scored evaluations of motor functioning based on clinical trial data (a higher score being associated with greater efficacy). In this example, the UPDRS Part III pharmaceutical parameter values are compared against an efficacy threshold level based on an average UPDRS Part III score for standard of care values for standard of care drugs that have been approved for human trials.

In this example, the UPDRS Part III pharmaceutical parameter values exceed the efficacy requirement threshold and the pharmaceutical assessment computing device 12 then determines whether the safety level requirement is satisfied. The pharmaceutical assessment computing device 12 may determine when a safety level threshold is exceeded, based on a comparison between one or more pharmaceutical parameter values and a safety threshold value. Here, the safety threshold is determined to be the highest tolerable dose of the drug candidate that is needed to achieve the desired therapeutic benefit. In this example, the pharmaceutical parameter value for daily dosage of the levodopa derivative is 5000 mg levodopa derivative per day which is less than the upper limit of the safety level threshold of 6000 mg per day of the candidate drug, beyond which the therapeutic benefits are outweighed by health risks associated with the drug candidate. Hence, the candidate drug satisfies the safety requirement in this example. If both the efficacy level requirement and the safety level requirement are satisfied, then the pharmaceutical assessment computing device 12 takes the Yes branch to step 318 in which a check for target validation data is performed.

If the efficacy level requirement or the safety level requirement is not satisfied in step 314 then the No branch is taken to step 316, and the pharmaceutical assessment computing device 12 sends a notification to client computing devices 14(1)-14(n) to indicate that the efficacy requirement or the safety level requirement is not satisfied. In alternative examples, the notification that the efficacy requirement or safety requirement is not satisfied may be provided through a different form of communication such as being included in a target product profile for the candidate drug.

In step 318, the pharmaceutical assessment computing device 12 determines whether target validation data is available by accessing server computing device 16(1)-16(n) and checking for target validation data. When no target validation data is available, then the No branch is taken to step 322, in which a target product profile is provided. In this example, the target validation data includes data based on animal model data, pharmacogenetic validation data, and supporting literature data.

The animal data includes efficacy based data on animal studies used to assess the effects of the candidate drug on an animal, including acute toxicity, sub-acute toxicity, off-target effects, ADME (Absorption Distribution Metabolism and Excretion) data, and pharmacodynamic data for drug affinity and potency.

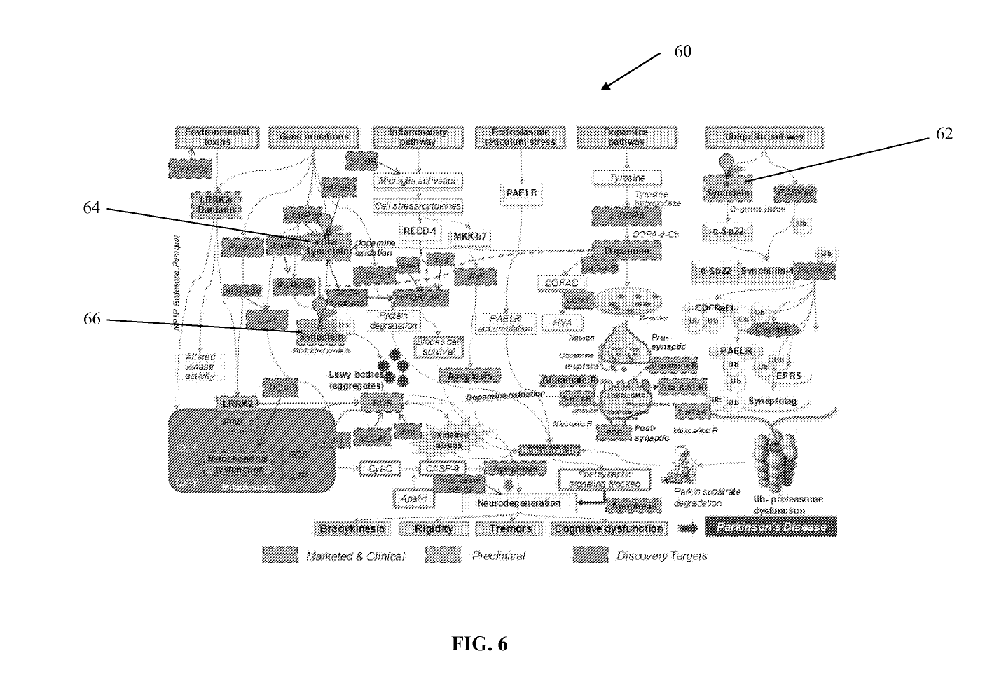

The pharmacogenetic validation data comprises functional validation data based on a model of the Parkinson's disease process as illustrated in FIG. 6 which shows a mapping of the alpha-synuclein 64 and alpha-synuclein 66 under the gene mutations section and alpha-synuclein 62 at the start of the Ubiquitin pathway.

The supporting literature data includes data from peer reviewed journals, key opinion leader data, though in an alternative example, the supporting literature may also include data from other sources such as non-peer reviewed journals and press releases.

If in step 318, target validation data is available, then the Yes branch is taken to step 320 and weighting factors are applied to the plurality of pharmaceutical data values. The weighting factors adjust the plurality of pharmaceutical data values based on the available target validation data. In this example, the target validation data is available in the form of pharmacogenetic data indicating the latest results of genetic biomarker data based on preclinical data indicating the greater significance of alpha-synuclein 64 and alpha-synuclein 66 in the treatment of Parkinson's disease. Accordingly, pharmaceutical assessment scores associated with genetic biomarkers are weighted more heavily than other pharmaceutical assessment scores. The weighting factor applied to the pharmaceutical assessment scores adjusts the weight of one or more pharmaceutical assessment scores relative to the remaining pharmaceutical assessment scores.

In an alternative example the weighting factor may include a standard deviation applied to one or more pharmaceutical assessment scores, a value that increases or decreases the one or more pharmaceutical assessment scores by a fixed amount, or a value such as a percentile value to adjust the weight of one or more pharmaceutical assessment scores.

In step 322, the pharmaceutical assessment computing device 12 provides a target product profile based on the assigned plurality of pharmaceutical assessment scores. In this example, the pharmaceutical assessment computing device 12 generates a target product profile including parameters for patient segment, efficacy, a safety profile and tolerability, dosage and administration, market access, maximal cost per dose, and upsides related to the candidate drug.

The patient segment parameter indicates the profile of a patient for whom the drug candidate is intended. The efficacy parameters indicate the effect of the candidate drug on the underlying disease process (Parkinson's) including primary endpoints such as the reduction in the UPDRS part III from a baseline level and secondary endpoints including reductions in the UPDRS part II from a baseline level. The safety profile and tolerability parameters disclose whether there are short-term or long term safety contraindications related to the candidate drug as well as data relating to patient tolerability of the candidate drug such as postural hypotension. The dosage and administration parameters include an indication of how the candidate drug should be administered (orally) and the recommended frequency of drug administration (more than once daily).

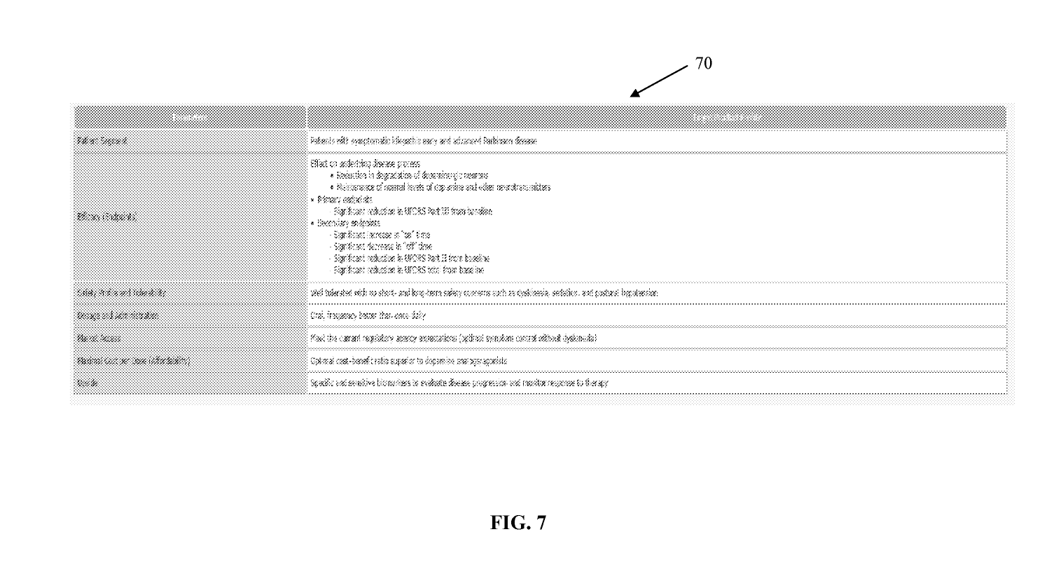

An example of a target product profile is illustrated in FIG. 7, which shows parameters for patient segment, efficacy, a safety profile and tolerability, dosage and administration, market access, maximal cost per dose, and upsides related to the candidate drug. After generating the target product profile, pharmaceutical assessment computing device 12 provides the target product profile to client computing devices 14(1)-14(n), though the target product profile may be communicated in different ways to different devices. In an alternative example, the target product profile may include any combination of pharmaceutical data and may be presented as part of a dashboard to facilitate saving the target product profile for later access.

In step 324, the pharmaceutical assessment computing device 12 identifies any therapy gaps in the pharmaceutical data based on the determined deviation of the pharmaceutical parameter values from the corresponding standard of care values. The therapy gaps are those areas of treatment that are not addressed by the target product profile in which the pharmaceutical assessment computing device 12 identifies standard of care values that exceed pharmaceutical parameter values by a predetermined therapy gap threshold value. In this example, the aggregated plurality of efficacy pharmaceutical parameter values for the candidate drug are less than half (0.3) the aggregated plurality of efficacy standard of care values which exceeds the therapy gap threshold value of 0.7 (meaning the pharmaceutical parameter values must be at least 70% of the value of the standard of care values). In an alternative example, the threshold gaps may be used as a basis for a weighting factor or provided in the target product profile.

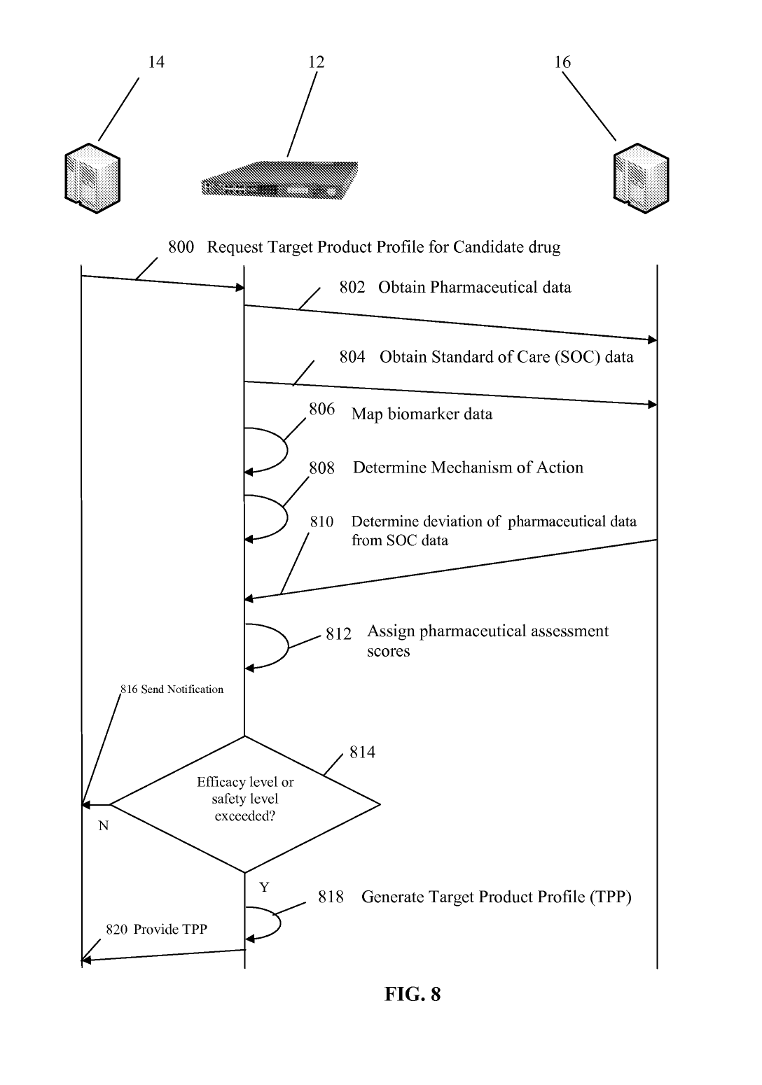

Referring more specifically to FIG. 8, a timing diagram illustrating an exemplary method of assessing pharmaceutical data is shown. In step 800, client computing devices 14(1)-14(n) send a request for a target product profile of a drug candidate to pharmaceutical assessment computing device 12. In this example, the request is a TCP/IP (Transmission Control Protocol/Internet Protocol) request, though other communications protocols may be used.

In step 802 pharmaceutical assessment computing device 12 obtains pharmaceutical data from server device 16(1)-16(n) by sending one or more requests for pharmaceutical data to server device 16(1)-16(n), which responds by sending the pharmaceutical data to pharmaceutical assessment computing device 12.

In step 804, pharmaceutical assessment computing device 12 obtains standard of care data from server devices 16(1)-16(n) by sending one or more requests for standard of care data to server devices 16(1)-16(n), which respond by sending the standard of care data to pharmaceutical assessment computing device 12.

In step 806, the pharmaceutical assessment computing device 12 maps biomarker data from the pharmaceutical data to disease indication targets and in step 808 determines a mechanism of action, based on the mapped biomarker data.

In step 810, the pharmaceutical assessment computing device 12 compares the pharmaceutical data to the standard of care data and in step 812 the pharmaceutical assessment computing device 12 assigns pharmaceutical scores to each one of the pharmaceutical parameters in the pharmaceutical data.

In step 814, the pharmaceutical assessment computing device 12 determines whether a required efficacy level or required safety level for the candidate drug data satisfies a predetermined efficacy threshold or predetermined safety threshold. If either the efficacy threshold or the safety threshold is not satisfied then pharmaceutical assessment computing device 12 sends a message in step 816 to client computing devices 14(1)-14(n) as a notification that the required efficacy level or the required safety level was not satisfied.

If the required efficacy level or the required safety level is satisfied in step 814, then the pharmaceutical assessment computing device 12 proceeds to step 818 and generates a target product profile, based on the assigned pharmaceutical assessment scores.

In step 820 pharmaceutical assessment computing device 12 provides the generated target product profile by sending the target product profile data to client computing devices 14(1)-14(n).

Accordingly, as illustrated and described by way of the examples herein, this technology provides effective methods, a non-transitory computer readable medium, and devices to assess pharmaceutical data. With this technology, a candidate drug is analyzed and an estimate of clinical performance is determined based on preclinical data, thereby providing a cost-effective way to rapidly assess the potential performance of a candidate drug. Further, the disclosed technology also provides an estimate of the expected efficacy or safety level of the candidate drug with minimal use of computational resources and without the expense associated with clinical trials. Additionally, by comparing pharmaceutical data for the candidate drug against standard of care data, the disclosed technology is able to objectively assess the overall performance of the candidate drug and provide a target product profile to effectively determine the likelihood of the candidate drug achieving regulatory approval.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for assessing pharmaceutical performance, the method comprising:
obtaining, by a pharmaceutical assessment computing device, for a candidate drug, pharmaceutical data comprising a drug indication and a pharmaceutical parameter value for each of a plurality of pharmaceutical parameters, wherein the drug indication comprises a medical condition treatable by the candidate drug;
obtaining, by the pharmaceutical assessment computing device, for the drug indication, standard of care data comprising a standard of care value for each of the plurality of pharmaceutical parameters;
determining, by the pharmaceutical assessment computing device, a deviation of each of the pharmaceutical parameter values from the corresponding one of the standard of care values;
assigning, by the pharmaceutical assessment computing device, one of a plurality of pharmaceutical assessment scores to each of the plurality of pharmaceutical parameters based on each of the determined deviations; and
providing, by the pharmaceutical assessment computing device, target product profile data for the candidate drug based on the assigned plurality of pharmaceutical assessment scores.

2. The method according to claim 1, wherein the plurality of pharmaceutical parameters comprises at least one of an efficacy parameter or a safety level parameter and further comprises:
comparing, by the pharmaceutical assessment computing device, the pharmaceutical parameter value for the efficacy parameter to the pharmaceutical parameter value for a required efficacy parameter or the pharmaceutical parameter value for the safety level parameter to the pharmaceutical parameter value for a required safety level parameter
providing, by the pharmaceutical assessment computing device, an indication when the efficacy value exceeds the required efficacy value or the safety level value exceeds the required safety level value.

3. The method according to claim 1, wherein the pharmaceutical data further comprises biomarker data for one or more biomarkers in the candidate drug and a plurality of drug indication target parameters for the drug indication and further comprises:
mapping, by the pharmaceutical assessment computing device, each of the one or more biomarkers to one or more of the plurality of drug indication target parameters, wherein the provided target product profile further comprises the mapped one or more biomarkers.

4. The method according to claim 3, further comprising:
determining, by the pharmaceutical assessment computing device, a mechanism of action for the candidate drug based on the mapped one or more biomarkers; and
providing, by the pharmaceutical assessment computing device, the determined mechanism of action.

5. The method according to claim 1, further comprising:
estimating, by the pharmaceutical assessment computing device, clinical performance of the candidate drug based on preclinical data, wherein the provided target product profile is further based on the estimated clinical performance of the candidate drug.

6. The method according to claim 1, further comprising:
determining, by the pharmaceutical assessment computing device, a weighting factor based on target validation data; and
adjusting, by the pharmaceutical assessment computing device, each one of the plurality of pharmaceutical assessment scores based on the determined weighting factor.

7. The method according to claim 1, further comprising:
identifying, by the pharmaceutical assessment computing device, a therapy gap in the candidate drug based on the determined deviation of each of the pharmaceutical parameter values from the corresponding one of the standard of care values.

8. A pharmaceutical assessment computing device, comprising:
at least one processor;
a memory, wherein the memory is coupled to the processor which is configured to be capable of executing programmed instructions, which comprise the programmed instructions stored in the memory to:
obtain, for a candidate drug, pharmaceutical data comprising a drug indication and a pharmaceutical parameter value for each of a plurality of pharmaceutical parameters, wherein the drug indication comprises a medical condition treatable by the candidate drug;
obtain, for the drug indication, standard of care data comprising a standard of care value for each of the plurality of pharmaceutical parameters;
determine a deviation of each of the pharmaceutical parameter values from the corresponding one of the standard of care values;
assign one of a plurality of pharmaceutical assessment scores to each of the plurality of pharmaceutical parameters based on each of the determined deviations; and
provide target product profile data for the candidate drug based on the assigned plurality of pharmaceutical assessment scores.

9. The device according to claim 8, wherein the plurality of pharmaceutical parameters comprises at least one of an efficacy parameter or a safety level parameter and wherein the processor is further configured to be capable of executing programmed instructions comprising and stored in the memory to:
compare the pharmaceutical parameter value for the efficacy parameter to the pharmaceutical parameter value for a required efficacy parameter or the pharmaceutical parameter value for the safety level parameter to the pharmaceutical parameter value for a required safety level parameter
provide an indication when the efficacy value exceeds the required efficacy value or the safety level value exceeds the required safety level value.

10. The device according to claim 8, wherein the pharmaceutical data further comprises biomarker data for one or more biomarkers in the candidate drug and a plurality of drug indication target parameters for the drug indication and wherein the processor is further configured to be capable of executing programmed instructions comprising and stored in the memory to:
map each of the one or more biomarkers to one or more of the plurality of drug indication target parameters, wherein the provided target product profile further comprises the mapped one or more biomarkers.

11. The device according to claim 10, wherein the processor is further configured to be capable of executing programmed instructions comprising and stored in the memory to:
determine a mechanism of action for the candidate drug based on the mapped one or more biomarkers; and
provide the determined mechanism of action.

12. The device according to claim 8, wherein the processor is further configured to be capable of executing programmed instructions comprising and stored in the memory to:
estimate clinical performance of the candidate drug based on preclinical data, wherein the provided target product profile is further based on the estimated clinical performance of the candidate drug.

13. The device according to claim 8, wherein the processor is further configured to be capable of executing programmed instructions comprising and stored in the memory to:
determine a weighting factor based on target validation data; and
adjust each one of the plurality of pharmaceutical assessment scores based on the determined weighting factor.

14. The device according to claim 8, further comprising:
identify a therapy gap in the candidate drug based on the determined deviation of each of the pharmaceutical parameter values from the corresponding one of the standard of care values.

15. A non-transitory computer readable medium having stored thereon instructions for assessing pharmaceutical data, comprising machine executable code which when executed by at least one processor, causes the processor to perform steps comprising:
obtaining, for a candidate drug, pharmaceutical data comprising a drug indication and a pharmaceutical parameter value for each of a plurality of pharmaceutical parameters, wherein the drug indication comprises a medical condition treatable by the candidate drug;
obtaining, for the drug indication, standard of care data comprising a standard of care value for each of the plurality of pharmaceutical parameters;
determining a deviation of each of the pharmaceutical parameter values from the corresponding one of the standard of care values;
assigning one of a plurality of pharmaceutical assessment scores to each of the plurality of pharmaceutical parameters based on each of the determined deviations; and
providing target product profile data for the candidate drug based on the assigned plurality of pharmaceutical assessment scores.

16. The medium according to claim 15, wherein the plurality of pharmaceutical parameters comprises at least one of an efficacy parameter or a safety level parameter, and further having stored thereon instructions that when executed by the processor cause the processor to perform steps further comprising:
comparing the pharmaceutical parameter value for the efficacy parameter to the pharmaceutical parameter value for a required efficacy parameter or the pharmaceutical parameter value for the safety level parameter to the pharmaceutical parameter value for a required safety level parameter
providing an indication when the efficacy value exceeds the required efficacy value or the safety level value exceeds the required safety level value.

17. The medium according to claim 15, wherein the pharmaceutical data further comprises biomarker data for one or more biomarkers in the candidate drug and a plurality of drug indication target parameters for the drug indication and further having stored thereon instructions that when executed by the processor cause the processor to perform steps further comprising:
mapping each of the one or more biomarkers to one or more of the plurality of drug indication target parameters, wherein the provided target product profile further comprises the mapped one or more biomarkers.

18. The medium according to claim 17, further having stored thereon instructions that when executed by the processor cause the processor to perform steps further comprising:
determining a mechanism of action for the candidate drug based on the mapped one or more biomarkers; and
providing the determined mechanism of action.

19. The medium according to claim 15, further having stored thereon instructions that when executed by the processor cause the processor to perform steps further comprising:
estimating clinical performance of the candidate drug based on preclinical data, wherein the provided target product profile is further based on the estimated clinical performance of the candidate drug.

20. The medium according to claim 15, further having stored thereon instructions that when executed by the processor cause the processor to perform steps further comprising:
determining a weighting factor based on target validation data; and
adjusting each one of the plurality of pharmaceutical assessment scores based on the determined weighting factor.

21. The medium according to claim 15, further comprising:
identifying a therapy gap for the candidate drug based on the determined deviation of each of the pharmaceutical parameter values from the corresponding one of the standard of care values.
